(19) United States
(12) Patent Application Publication
(10) Pub. No.: US 2003/0232384 A1
(43) Pub. Date: Dec. 18, 2003

(54) Microarray system utilizing microtiter plates

(75) Inventors: Thomas E. Kocher, Rochester, NY (US); Timothy J. Wojcik, Rochester, NY (US)

Correspondence Address: Thomas H. Close, Patent Legal Staff, Eastman Kodak Company, 343 State Street, Rochester, NY, 14650-2201, US

(73) Assignee: Eastman Kodak Company

(21) Appl. No.: 10/171,169

(22) Filed: Jun. 13, 2002

(51) Int. Cl. G01N 33/53; C12M 1/34

(52) U.S. Cl. 435/7.1; 435/287.2

(57) ABSTRACT

A biological analysis system comprising: a microarray receiver including a substrate coated with a composition including a population of biological probe modified micro-spheres immobilized in a coating containing a gelling agent or precursor to a gelling agent, at least a sub-population of the population of micro-spheres containing an optical bar code generated from at least one colorant associated with the micro-spheres and including a biological probe, and a microtiter plate having a plurality of wells open at first and second ends in liquid sealing contact with the microarray receiver at the first open ends, each of the wells adapted to receive a fluorescently/chemiluminescently labeled biological target sample which interacts fluorescently/chemiluminescently with the biological probe.

### FIELD OF THE INVENTION

This invention relates in general to a biological analysis system and more particularly to a biological analysis system which integrates microarray receivers with microtiter plates.

### BACKGROUND OF THE INVENTION

In recent years, high density arrays formed by spatially addressable bioactive probes on a receiver has greatly enhanced and simplified the process of biological research and development. More specifically, the molecular biology community has developed considerable infrastructure based on microtiter plate technology. Microtiter plates are devices that contain wells that enable molecular biological experiments by combining several chemistries confined in wells. An example of microtiter plates can be found with many manufacturers, including Whatman Philtronics as well as Corning, Inc., to name a few. Microtiter plates are available in many configurations including 96 wells, 192 wells, 384 wells, etc. Plates are available with round wells, square wells, and with filters in the bottom, for example. Since microtiter plate technology has been so widely adopted by the molecular biology community as well as the drug discovery and the drug development communities, a significant amount of robotics technology has been developed to automate the processes that use microtiter plates. In general, robotics are used to move plates from location to location as well as dispense liquids into the wells. Automation provides speed as well as consistency. Array-based ELISAs for High-Throughput Analysis of Human Cytokines, BioTechniques, Vol. 31, No. 1 (2201), 186-194, (Pierce Endogen) describes a technique where antibody solutions at 20 nL/spot were spotted in a 3×3 pattern in well plates. High-Throughput micorarray-based enzyme-Linked Immunosorbent Assay (ELISA), BioTechniques, Vol. 27, No. 4, (1999), 778-788, (Genometrix) demonstrates the potential to conduct multi-analyte assays using 96 well microarray-based ELISA format.

The Corning/Life Sciences web site has extensive information on products used in microtiter plate assay systems. Problems with this technology are the expense and complexity of equipment for forming the biosite arrays in the wells of the microtiter plates.

The following patents disclose alternative microarray techniques that do not adequately solve these problems. U.S. Pat. No. 6,083,763, issued Jul. 4, 2000, inventor Balch, discloses in FIG. 7, a thin film substrate having conductive patterns and biosites deposited thereon which are bonded to a reaction vessel having a matrix of open reaction chambers that are closed off by the thin film substrate. The biosites are disclosed as being deposited by ink-jet, capillary, or photolithiographic techniques. It is also disclosed to bond thin films of plastic or glass to conventional bottomless microtiter plates. Biosites are deposited either before or after the bottoms are bonded to the plate.

U.S. Pat. No. 6,232,066, issued May 15, 2001, inventors Felder et al., discloses a multi-well assemblage including a well separator, a subdivider, and a base which are joined together. Biosite arrays are attached to the base.

U.S. Pat. No. 6,309,828, issued Oct. 30, 2001, inventors Schleifer et al., discloses a technique for applying an array of nucleic acid molecules on a substrate by a device including a synthesis unit in communication with a purification unit in communication with a printing unit. The synthesis, purification, and printing units, are aligned, bottomless microtiter plates.

### SUMMARY OF THE INVENTION

According to the present invention, there is provided a solution to the problems discussed above.

According to a feature of the present invention, there is provided a biological analysis system comprising: a microarray receiver including a substrate coated with a composition including a population of biological probe modified micro-spheres immobilized in a coating containing a gelling agent or precursor to a gelling agent, at least a sub-population of said population of micro-spheres containing an optical bar code generated from a least one colorant associated with the micro-spheres and including a biological probe; and a microtiter plate having a plurality of wells open at first and second ends in liquid sealing contact with said microarray receiver at said first open ends, each of said wells adapted to receive a fluorescently/chemiluminescently labeled biological target sample which interacts fluorescently/chemiluminescently with said biological probe.

### ADVANTAGEOUS EFFECT OF THE INVENTION

The invention has the following advantages.

1. A system is provided incorporating microtiter plates and microarray receivers which is simple and cost efficient.

2. The microarray receivers can be either patterned or random arrays of biological probes.

3. The receiver can be opaque or transparent to facilitate detection by reflection emission or transmission detection devices.

### BRIEF DESCRIPTION OF THE DRAWINGS

FIG. 1 is a partial elevational diagrammatic view illustrating an embodiment of the invention.

FIG. 2 is a perspective diagrammatic view illustrating the embodiment of FIG. 1.

FIGS. 3A and 3B are diagrammatic views of microarray receivers showing respectively patterned and randomly distributed biologically active sites.

FIGS. 4A-4C are respective diagrammatic views of optical readout systems for use in conjunction with the present invention.

FIG. 5 is a diagrammatic view of a microarray receiver which can be used in the present invention.

### DETAILED DESCRIPTION OF THE INVENTION

In general, according to the present invention there is provided a biological analysis system including the combination of a microtiter plate having an array of open-ended wells and a microarray receiver having random or predetermined array of biologically functional sites which form a repetitive pattern on the receiver and which are in contact with the open ended wells. The microarray receiver is described in U.S. patent application Ser. No. 09/942,241, the contents of which are hereby incorporated by reference. A general description of the microarray receiver will now be given but reference is made to the latter patent application for a more complete description.

The microarray receiver according to the invention, includes a substrate coated with a composition comprising micro-spheres (beads) dispersed in a fluid containing a gelling agent or a precursor to a gelling agent, wherein the micro-spheres are immobilized in a random or ordered position on the substrate. The substrate is free of receptors designed to physically or chemically interact with the micro-spheres. One or more sub-populations of the population of microspheres contain a unique optical bar code generated from at least one colorant associated with the micro-spheres and including a unique biological functionality or probe which react with analytes with which they come in contact.

The distribution or pattern of micro-spheres on the substrate may be entirely random (a spatial distribution showing no reference or bias) or be attracted or held to sites that are pre-marked or predetermined on the substrate. Each micro-sphere in the array has a distinct signature based on color which may be derived from mixing three dyes representing the primary colors Red (R), Green (G), and Blue (B) to create thousands of distinguishable micro-spheres with distinct color addresses (unique RGB values, e.g., R=0, G=204, B=153). The micro-spheres are made with active sites on their surface to which are attached a specific bioactive probe. Therefore, each color address can correspond to a specific bioactive probe.

A microarray or population of micro-spheres can include a few or hundreds or more of sub-populations of micro-spheres, where each sub-population comprises the same color code and the same bio-active probe. Each microarray of micro-spheres occupies a sub-area of the substrate and is repeated in a pattern over the area of the substrate. The dimensional area of the microarray sub-area may be comparable to the dimensional area of a microtiter well or multiple wells may overlay a microarray sub-area.

The micro-spheres are preferably coated onto the substrate as disclosed in U.S. patent application Ser. No. 09/942,241.

In order to use a microarray having bioactive probes to analyze an unknown biological target sample, the sample to be analyzed has to be nonselectively labeled by using fluorescent dyes or chemiluminescent active molecules.

A biological target sample placed into a microtiter plate well comes into contact with the microarray bioactive probes. The fluorescently/chemiluminescently signals which result from the hybridization of the unknown biological target sample with bioactive probes on the surface of the coated micro-spheres are detected and analyzed by an electronic camera/image processor system.

Microtiter plates are available in many configurations including 96 wells, 192 wells, 384 wells, etc. Plates are available with round and square wells and with filters in the bottom.

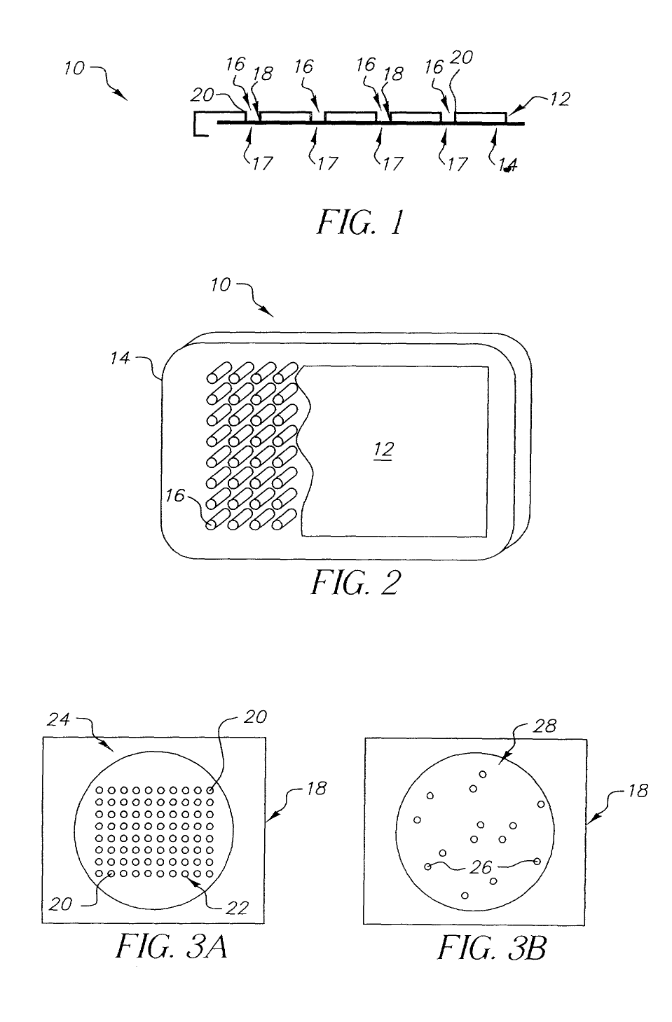

Referring now to FIG. 1, there is shown an embodiment of the present invention. As shown, biological analysis system 10 includes microtiter plate 12 with microarray receiver 14 integrated to the bottom of plate 12. Plate 12 and receiver 14 are shown in partial cross section. Plate 12 includes open-ended wells 16 having openings at first and second ends 18, 20. Microarray receiver 14 with areas of biological functionality 17 is in sealing contact with first ends 18 of wells 16. Preferably microarray receiver 14 has had its biological function established either through ordered deposition or random coating techniques before being combined with plate 12. In an alternative embodiment, the receiver is sealed to the bottom of plate 12 prior to providing biological functionality which is provided through wells 16 or plate 12 by well known techniques.

FIG. 2 shows the system 10 from the bottom. Microarray receiver 12 has been partially removed to display wells 16 in microtiter plate 14. Receiver 12 normally extends over the entire bottom of plate 14 covering all wells 16. In use, the wells 16 are filled with a sample biological target analyte.

FIG. 3A shows a region 18 of a microarray receiver showing sites 20 with biological functionality. The sites 20 are arranged in a predetermined patterned array 22. Side wall 24 of a well is shown bounding site array 22.

FIG. 3B shows a region 18 of a microarray receiver showing sites 26 with biological functionality. The sites 26 are arranged in a random array 28. Side wall 24 of a well is shown bounding site array 28.

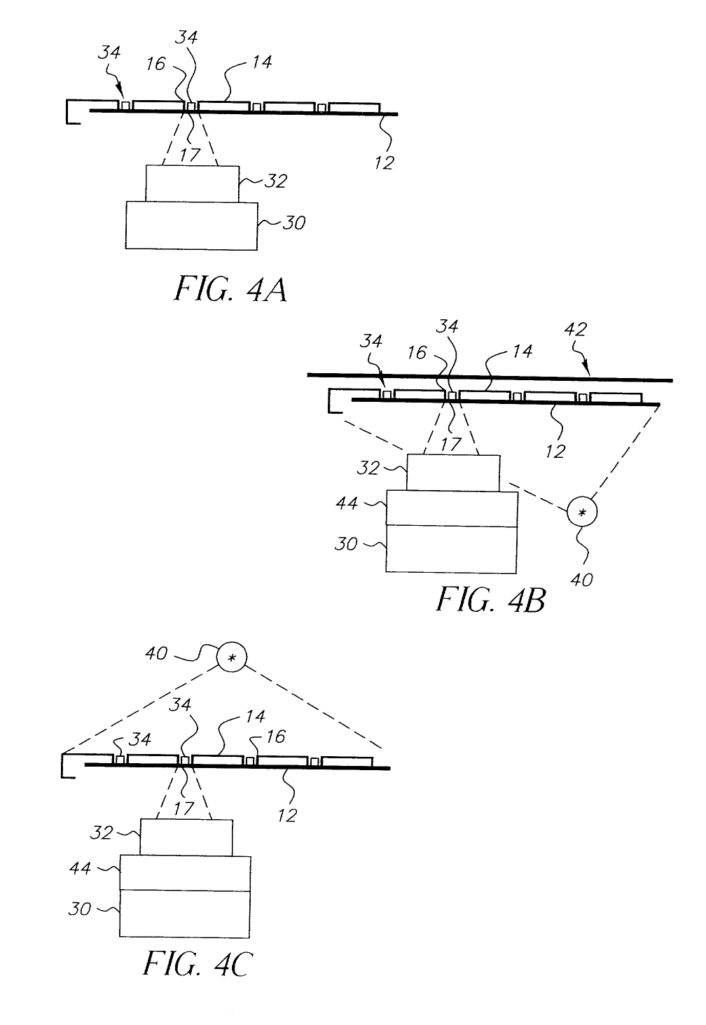

Referring now to FIGS. 4A, 4B, 4C, there are shown optical readout systems for luminescence, reflection fluorescence and transmission fluorescence, respectively. As shown in FIG. 4A, the hybridization of a target analyte 34 in a well 16 of plate 14 with a biological probe 17 on microarray receiver 12 produces luminescence which is detected by CCD camera 30 with lens 32.

As shown in FIG. 4B, a source of illumination 40 illuminates the backside of microarray receiver 12. The hybridization of target analyte 34 with a biological probe 17 fluoresces and the emissions are reflected from reflector 42 to camera 30 with lens 32. Filter 44 filters out the illuminant light while passing the fluorescent light.

As shown in FIG. 4C, a source of illumination 50 illuminates analytes 34 from the front side of plate 14 and receiver 12. Since receiver 12 in this case is light transmissive, the fluorescent light emissions from the hybridization of analyte 34 with a biological probe 17 pass through receiver 12 to CCD camera 30 with lens 32 and filter 44, which filters out the illuminant light but passes the fluorescent light.

It will be understood that the components used in the optical readout systems of FIGS. 4A-4C can be other than those shown. For example, electronic or digital cameras with sensors other than CCD can be used. In addition, the positioning of the camera can result in more complex optical systems than those shown.

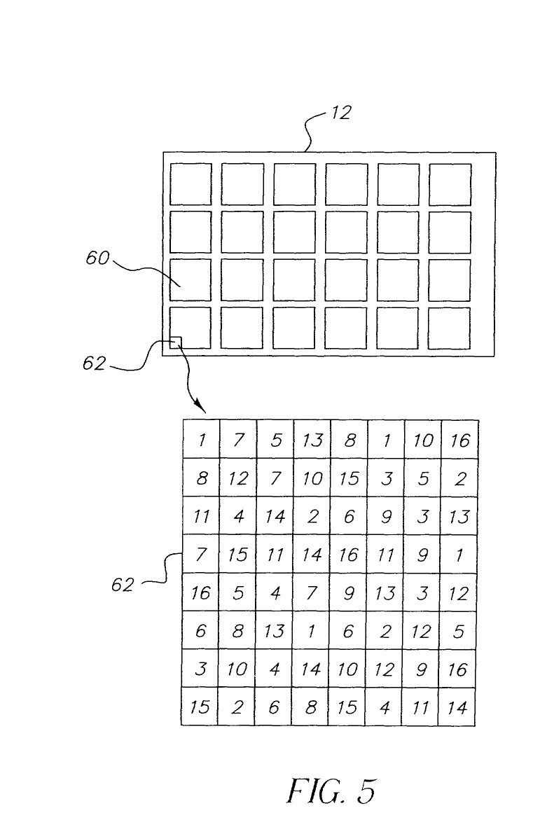

FIG. 5 illustrates a microarray receiver that can be used in the present invention. As shown, microarray receiver 12 includes a pattern of 24 regions 60 in a matrix of 4 rows and 6 columns. Each region includes an identical microarray of randomly distributed biological probe sites, a portion of which are shown in the exploded view. In this view, 16 different biological probes attached to micro-spheres are randomly distributed throughout the portion 62 of region 60. According to the invention, each probe is attached to a micro-sphere of a color unique to that probe so that micro-spheres of 16 different colors are present in portion 16. If, for example, an analyte containing each of the 16 complimentary targets to the 16 probes is brought into contact with portion 62, the hybridization between the 16 targets with the 16 probes would produce luminescence or fluorescence of 16 different colors which are detected by an appropriate optical system (FIGS. 4A-4C).

The invention has been described in detail with particular reference to certain preferred embodiments thereof, but it will be understood that variations and modifications can be effected within the spirit and scope of the invention.

### PARTS LIST

10 biological analysis system

12 microtiter plate

14 microarray receiver

16 open-ended wells

17 biological probe

18,20 first and second ends

22 patterned array

24 side wall

28 site array

30 CCD camera

32 lens

34 target analyte

40 illumination

42 reflector

44 filter

50 source of illumination

60 regions

62 portion

What is claimed is:

1. A biological analysis system comprising:
a microarray receiver including a substrate coated with a composition including a population of biological probe modified micro-spheres immobilized in a coating containing a gelling agent or precursor to a gelling agent, at least a sub-population of said population of micro-spheres containing an optical bar code generated from a least one colorant associated with the micro-spheres and including a biological probe; and
a microtiter plate having a plurality of wells open at first and second ends in liquid sealing contact with said microarray receiver at said first open ends, each of said wells adapted to receive a fluorescently/chemiluminescently labeled biological target sample which interacts fluorescently/chemiluminescently with said biological probe.

2. The system of claim 1 wherein said microarray receiver substrate is coated with a composition including a population of biological probe modified micro-spheres immobilized in a coating containing a gelling agent or precursor to a gelling agent, said population including a plurality of sub-populations of micro-spheres, each of which contains a unique bar code generated from at least one colorant associated with said micro-spheres and including a unique biological probe.

3. The system of claim 1 wherein said microarray receiver includes a pattern of regions, each of which includes an identical population of said micro-spheres.

4. The system of claim 3 wherein said population of microspheres in each of said regions includes a plurality of sub-populations of microspheres, each of which contains a unique bar code generated from at least one colorant associated with said micro-spheres and including a unique biological probe.

5. The system of claim 3 wherein at least one microtiter well is in contact with each region of said pattern of regions.

6. The system of claim 3 wherein two or more microtiter wells are in contact with each region of said pattern of regions.

7. The system of claim 1 wherein said optical barcode is generated by two or more colorants.

8. The system of claim 1 wherein said optical barcode is generated by a mixture of red (R), green (G), and blue (B) colorants.
